(19) United States
(12) Patent Application Publication
(10) Pub. No.: US 2024/0335397 A1
(43) Pub. Date: Oct. 10, 2024

(54) USE OF POLYAMINES IN THE TREATMENT OF BRAIN TUMOURS

(71) Applicant: CAMBRIDGE ENTERPRISE LIMITED, Cambridge Cambridgeshire (GB)

(72) Inventors: Melinda Jane DUER, Cambridge Cambridgeshire (GB); Uliana BASHTANOVA, Girton Cambridge Cambridgeshire (GB)

(21) Appl. No.: 18/682,559

(22) PCT Filed: Aug. 10, 2022
(86) PCT No.: PCT/GB2022/052082

§ 371 Date: Feb. 9, 2024

(30) Foreign Application Priority Data
Aug. 11, 2021 (GB) 2111547.2

(51) Int. Cl. A61K 31/132; A61K 31/722; A61K 31/785; A61P 35/00

(52) U.S. Cl. CPC A61K 31/132; A61K 31/722; A61K 31/785; A61P 35/00

(57) ABSTRACT

The present invention relates to the field of brain tumour treatment. The invention provides methods of killing brain tumour cells in these tissues using high concentrations of cytotoxic agents which are capable of binding strongly to normal brain extracellular matrix (ECM), but less strongly to extracellular matrix components in the tumour environment. The cytotoxic agent is a polyamine, e.g. spermidine or putrescine.

The present invention relates to the field of brain tumour treatment. The invention provides methods of killing brain tumour cells in brain tumours using high concentrations of cytotoxic agents which are capable of binding strongly to normal brain extracellular matrix (ECM), but less strongly to extracellular matrix components in the tumour environment. The cytotoxic agent is a polyamine, e.g. spermidine or putrescine.

Brain cancers, exemplified by glioblastoma multiforme (GBM), are particularly difficult to treat [M. Monticelli et al., Clinical Neurol. Neurosurg. 170 (2018) 120-126]. Very few anti-cancer drugs are able to cross the blood-brain barrier in any significant concentration, so current standard of care for primary GBM tumours begins with tumour resection. The diffuse nature of GBM tumour margins means that it is impossible to remove all cancerous cells during surgery. Thus, GBM tumours cannot be cured by surgery, and tumour regrowth after surgery at the original site and metastasis to other areas of the brain is inevitable. Metastasis is most commonly to brain regions within 2 cm of the original tumour body as determined by MRI, but can include more distant metastases to, for example, the contralateral hemisphere.

The current standard-of-care post-surgery, or in cases where surgery is not warranted, is therefore radiotherapy to a field incorporating the primary tumour site with a 2-3 cm margin and concurrent, adjuvant chemotherapy. These therapies aim to slow tumour recurrence and metastasis, but again, are not curative. Oronsky et al's [B. Oronsky et al., Frontier in Oncology, 10 (2021) 574012] review of newly-diagnosed GBM tumours provides a comprehensive picture of the current standard of care for GBM, including discussion of clinically-relevant biomarkers.

Radiation treatment, whilst essential in the current standard of care for GBM, unfortunately leads to long term severe impact on brain function for longer-lived patients [Y. W. Lee et al., Biomol. Ther. 20 (2012) 357-370; C. Turnquist et al., Neuro-Oncology Advances, 2 (2020) vdaa057].

Temozolomide, a DNA alkylating agent, is the current standard-of-care adjuvant chemotherapy drug in GBM that is used concurrently with radiation treatment. DNA-alkylating nitrosoureas, Lomustine (CCNU) and Carmustine (BCNU or BiCNU) are also used in GBM treatment, though currently less commonly than Temozolomide. Carmustine can be delivered via Gliadel® wafers placed into the tumour resection cavity. Lomustine shows therapeutic activity in patients with O⁶-methylguanine DNA methyltransferase (MGMT) promoter methylation [M. Weller, E. Le Rhun, Cancer Treatment Rev. 87 (2020) 102029] when used alone and is also used as part of multidrug treatment programs (with Procarbazine and Vincristin). However, the blood-brain barrier always limits the concentration of chemotherapy drugs that reach brain cancer cells. Possibly because of this, drug resistance is a common feature in GBM. Thus, despite aggressive treatment with maximal tumour resection followed by radiation plus chemotherapy, median GBM patient survival is currently only 14-16 months from diagnosis for this standard of care.

Bevacizumab is a monoclonal antibody against vascular endothelial growth factor (VEGF) that aims to inhibit vascularization of the tumour [O. D. Arevalo et al., Frontiers in Neurology, 10 (2019)]. GBM tumours are highly hypoxic environments and neovascularization is essential for tumour progression [B. Oronsky et al., Frontiers in Oncology, 10 (2021) 574012]. Phase III trials with Bevacizumab as a first-line therapy demonstrated an increase in median progression-free survival, but not in overall survival. Thus, Bevacizumab is licensed in some countries for use in recurrent GBM, but not as a first-line therapy. Bevacizumab in combination with Lomustine has shown increases in median progression-free survival (+0.23 months) and overall survival (+1.4 months) in patients with recurrent GBM [Ren et al., Frontiers in Neurology, 11 (2021) 603947], but these are not large increases in survival time.

An alternative recent advance in GBM treatment uses so-called Tumour Treatment Fields, alternating electric fields delivered by a close-fitting cap over the scalp, via the Optune® device, to disrupt cancer cell division [D. Fabian et al., Cancers 11 (2019) 174]. The US FDA approved Optune® for adjuvant use with temozolomide in first-line GBM treatment in 2015 after interim results in a Phase III trial showed progression-free survival was 19.6 months with this combination compared to 16.6 months with temozolomide alone.

The treatment options for GBM thus remain limited especially compared to other solid cancers, and prognosis is, in all cases, poor whatever the treatment. Even with multimodal intervention, recent studies show that median survival for patients is still only 14-16 months with 26-33% surviving to 2 years and 5% to 5 years [M. R. Gilbert, J Clin Oncol. 31 (2013) 4085-91; B. Oronsky et al., Frontiers in Oncology, 10 (2021) 574012].

With such short overall survival times, the adverse effects of radiation therapy on brain function are accepted by patients and those treating them, because most patients do not live long enough for these longer-term effects to become apparent. However, if long term survival of GBM patients becomes a realistic prospect, there is considerable need for approaches with minimal side-effects on normal brain tissue. There is thus a significant clinical unmet need for new approaches to both extend progression-free and overall survival for GBM patients, and to improve patient quality of life in the longer term.

Brain metastasis-secondary tumours forming in the brain as a result of cancer cell invasion from tumours in other tissues-occur in ˜15% of cancer patients and is particularly common in patients with primary breast or lung cancer or melanoma. The tumour environment in brain metastatic cancer has many similarities with primary brain cancers and the blood-brain barrier similarly severely limits chemotherapy treatment options [A. Boire et al., Nature Cancer Rev. 20 (2020) 4-11]. Surgical resection is rarely curative and only patients with a single brain lesion are considered for surgery. Whole brain irradiation is the other main treatment applied. However, even with treatment, medial survival time for patients with brain metastasis is less than 6 months. There is thus a huge unmet need for new therapeutic strategies for metastatic brain cancer as well as primary brain tumours.

The inventors have developed a new approach to treating brain tumours, including GBM tumours and other brain cancers, and metastatic brain disease by exploiting inherent differences between tumour and normal brain extracellular matrix (ECM) to selectively kill tumour cells.

This selectivity was achieved by discovering drug molecules that are strongly sequestered by normal brain ECM, but only weakly by the tumour ECM. This results in the concentration of available free (unbound) drug in normal brain ECM being too low to be cytotoxic (drug concentration below the EC₅₀), whilst in the tumour environment, the free drug concentration can be sufficiently high to kill cells (concentration above the EC₅₀). Thus, very high concentrations of these drugs may be delivered directly to the tumour, for instance during surgical resection or intra-tumoral infusion, to rapidly kill cells in the tumour whilst having relatively few side-effects on normal brain tissue.

The differences in normal brain and brain tumour ECM that the inventors exploit are two-fold: differences in composition of the ECM; and differences in the volume of ECM relative to the volume of cells in the two environments.

The most abundant single component of normal brain extracellular matrix is the polyanion, hyaluronic acid (10 wt % by dry weight), followed by proteoglycans (15 wt %; (lecticans (aggrecan, versican, neurocan and brevican) and others (decorin, biglycan, phosphacan)) that are also necessarily negatively-charged by virtue of their glycosaminoglycan polyanion post-translational modifications. The brain tumour extracellular environment is by contrast, rich in more hydrophobic proteins such as fibronectin and collagens.

The inventors have found that positively-charged drug molecules bind strongly to normal brain ECM components, and less strongly to tumour ECM, because of the relatively high abundance of negatively-charged macromolecules in normal brain ECM compared with tumour ECM. They therefore found that candidate drug molecules for achieving the desired cell-killing selectivity based on ECM composition were positively-charged molecules.

Additionally, the volume of ECM/cell ratio in brain tumours is much lower than in normal brain tissue, the tumour being a cell-crowded environment compared to normal brain tissue, i.e. there is significantly less ECM between cells in brain tumours compared to normal brain tissue. Thus, the inventors have found that the concentration of unbound, active drug can remain high in the tumour environment, even where the tumour ECM has binding affinity for the drug.

Moreover, the inventors reasoned that positively-charged drugs would have limited diffusion within the normal brain extracellular matrix because of their strong binding to the abundant hyaluronic acid and proteoglycans in that ECM. In contrast, the relative lack of binding to the tumour ECM suggested that diffusion of a charged drug molecule would be significantly more facile in the extracellular regions around and within the tumour. Thus, if injected into a brain tumour, positively-charged drug molecules can be expected to leach relatively limited distances into surrounding normal brain ECM, but diffuse more freely in the tumour environment, allowing the drug to come into contact with a significant proportion of the cancer cells.

The inventors had the insight to realise that one could exploit high positive charges on a drug molecule to kill cells. Larger polycations are known to punch holes in cell plasma membranes; this is hypothesised to be due to the polycation sequestering membrane anionic phospholipids.

This effect is used to assist delivery of DNA into cells for instance. At higher concentrations of polycation, cell death occurs because the high level of phospholipid sequestering by the polycation causes extensive membrane damage. Smaller polycations, e.g. spermine, are known to depolarize the cell plasma membrane, which causes water ingress and cell lysis/fragmentation. Both effects are efficient cell killing mechanisms.

The inventors realised that these mechanisms of cytotoxicity would be prevented if the polycation was sequestered into the extracellular matrix preferentially to binding cell membrane phospholipids. In other words, the inventors realised that if the chemical equilibrium of binding for the drug to ECM and cell membrane phospholipids favoured binding to the ECM in normal brain, but favoured binding to phospholipids in the tumour environment, that the drug would be selective for killing cells in the tumour and not in healthy tissue.

Polyamines are polycations and a number of polyamines are known to be cytotoxic if present in sufficient concentration. For instance, the cytotoxicity of polyamines such as PAMAMs has been hypothesised to be due to their positive charge disrupting the cell membrane by sequestering phospholipids, as described above.

Some polyamine drugs have been suggested before for use in treating cancers, but not by directly delivering a high concentration of them to cause very rapid cell death with little side-effects on the host tissue. Indeed, those skilled in the art would have expected such high doses to be lethal to the host regardless of the tissue into which they were injected.

It is shown herein, however, that the toxicity of high concentrations of a number of polyamines is quenched in a hyaluronic acid-rich environment, demonstrating that their toxicity would be similarly quenched in normal brain ECM.

There are several advantages to the invention, including the following:

(1) The ability to kill all cell types in the cancer environment. This includes heterogeneous populations of cancer cells, which normally pose a challenge for other treatment strategies. It may also include other cell types such as cancer-associated fibroblasts (CAFs) and microglia, which although not cancer cells, often contribute to tumour progression.

(2) The approach does not depend on particular cancer cell mutations and is thus especially advantageous in GBM where there is known to be considerable heterogeneity in cancer cell genotype that confounds current chemotherapy and radiation treatments.

(3) The method of killing cancer cells by the invention (i.e. by sequestering cell membrane phospholipids and/or binding to cell membrane phospholipids and thereby depolarizing the cell membrane) is unlikely to be particularly susceptible to drug resistance, because the drugs do not need to enter the cell to kill it. The sequestering of and binding to membrane phospholipids are physical processes, dependent only on the strength of chemical binding between phospholipid-drug, that will necessarily occur if the drug and cell membrane are in close proximity. Thus, these drugs are unlikely to be subject to the known drug-resistance pathways.

(4) The invention may also be used as an alternative to tumour resection for removing substantial portions of a brain tumour with fewer potential complications and causing less stress response than conventional surgical options.

It is one object of the invention, therefore, to provide a method for killing brain tumour cells whilst having little impact on cells in the normal brain environment. In particular, it is one object of the invention to kill cancer cells which might remain after the resection of a brain tumour.

Following surgical resection of brain glioblastoma multiforme, tumour regrowth is known to occur in nearly all patients at the original tumour site with 3-6 months; cells derived from this tumour also often invade other parts of the brain. It is therefore an object of the invention to provide methods to prevent such tumour regrowth or invasion in brain glioblastoma multiforme, and in other tumours.

In one embodiment, the invention provides an in vivo method of killing brain tumour cells in a brain tumour or at a site of a resected brain tumour in a subject, the method comprising the step of:
(a) contacting brain tumour cells in the brain tumour or at the site of the resected brain tumour with a composition comprising a cytotoxic agent,
   wherein the cytotoxic agent is one in which the chemical equilibrium between:
(i) cytotoxic agent bound to the ECM and
(ii) free, unbound cytotoxic agent
   favours the ECM-bound state in normal brain ECM, and favours the free, unbound state in the tumour ECM.

In another embodiment, the invention provides a cytotoxic agent for use in an in vivo method of killing brain tumour cells in a brain tumour or at a site of a resected brain tumour, the method preferably comprising the step of:
(a) contacting brain tumour cells in the brain tumour or at the site of the resected brain tumour with a composition comprising the cytotoxic agent,
   wherein the cytotoxic agent is one in which the chemical equilibrium between:
(i) cytotoxic agent bound to the ECM and
(ii) free, unbound cytotoxic agent
   favours the ECM-bound state in normal brain ECM, and favours the free, unbound state in tumour ECM.

In yet another embodiment, the invention provides the use of a cytotoxic agent in the manufacture of a medicament for use in a method of killing brain tumour cells in a brain tumour or at a site of a resected brain tumour, the method preferably comprising the step of
(a) contacting brain tumour cells in the brain tumour or at the site of the resected brain tumour with a composition comprising a cytotoxic agent,
   wherein the cytotoxic agent is one in which the chemical equilibrium between
(i) cytotoxic agent bound to the ECM, and
(ii) free, unbound cytotoxic agent,
   favours the ECM-bound state in normal brain ECM, and favours the free, unbound state in tumour ECM.

In yet another embodiment, the invention provides an in vivo method of killing brain tumour cells in a brain tumour or at a site of a resected brain tumour in a subject, the method comprising the step of:
(a) contacting brain tumour cells in the brain tumour or at the site of the resected brain tumour with a composition comprising a cytotoxic agent,
   wherein the cytotoxic agent is one which binds strongly to hyaluronic acid and/or brain extracellular matrix proteoglycans.

In yet another embodiment, the invention provides a cytotoxic agent for use in an in vivo method of killing brain tumour cells in a brain tumour or at a site of a resected brain tumour, the method preferably comprising the step of
(a) contacting brain tumour cells in the brain tumour or at the site of the resected brain tumour with a composition comprising a cytotoxic agent,
   wherein the cytotoxic agent is one which binds strongly to hyaluronic acid and/or brain extracellular matrix proteoglycans.

In yet another embodiment, the invention provides the of a cytotoxic agent in the manufacture of a medicament for use in an in vivo method of killing brain tumour cells in a brain tumour or at a site of a resected brain tumour, the method preferably comprising the step of
(a) contacting brain tumour cells in the brain tumour or at the site of the resected brain tumour with a composition comprising a cytotoxic agent,
   wherein the cytotoxic agent is one which binds strongly to hyaluronic acid and/or brain extracellular matrix proteoglycans.

The contacting is preferably directly contacting. The cytotoxic agent is preferably a polyamine.

The method of the invention is carried out in vivo, i.e. in the human or animal body. Preferably, the subject is a mammal, more preferably a human, mouse, rat, horse, pig, cow, sheep, goat. Most preferably, the subject is human. In some embodiments, the subject is a non-human mammal. The human may, for example, be 0-10, 10-20, 20-30, 30-40, 40-50, 50-60, 60-70, 70-80, 80-90, 90-100 or above 100 years old.

The brain tumour may be a benign, pre-malignant or malignant tumour. The brain tumour may be a primary or secondary tumour. The brain tumour is preferably a solid tumour. The brain tumour comprises brain tumour or brain cancer cells, and their metastatic expansion within the healthy brain tissue. The brain tumour may also include cancer-associated fibroblasts and immune cells, such as microglia.

In some embodiments, the tumour is one whose size or carcinogenic (e.g. invasive) capabilities has previously been reduced. For example, the tumour may be one which has previously been at least partially resected (removed). Alternatively, or additionally, the tumour may be one which has previously been treated with another anti-tumour treatment, e.g. chemotherapy, immunotherapy or radiation, or a combination thereof.

Normal brain parenchyma extracellular matrix (ECM) consists of hyaluronic acid, proteoglycans (lecticans (versican, neurocan and brevican) and others (decorin, biglycan, phosphacan)), link proteins, ITIH2 and tenascin-R (along with smaller amounts of other tenascins). Around vessels in the brain, the ECM contains fibrous glycoproteins, the most abundant of which are fibronectin and laminin, basement membrane proteins (primarily collagen IV and laminin, also proteoglycans, agrin and perlecan).

The composition of normal brain extracellular matrix overall by weight is approximately 10% hyaluronic acid: 15% proteoglycans (total comprising many different proteoglycans): 1% collagen IV by dry weight (K. Koh, J. Cha, J. Park, J. Choi, S.-G. Kang, P. Kim. Scientific Reports 8 (2018) 4608), corresponding to 3%: 4.5%: 0.3% by fresh weight, respectively, assuming 70% of brain tissue is water.

The most abundant single component of the normal brain extracellular matrix is thus the negatively-charged polyanion, hyaluronic acid (3 wet wt %), followed by proteoglycans that are also necessarily negatively-charged by virtue of their glycosaminoglycan post-translational modifications. Such components may therefore readily be bound by polycations, e.g. polyamines.

The ECM of GBM and metastatic tumours in the brain is typically enriched with proteins more associated with basal membrane than brain parenchyma matrix: fibronectin, collagen IV and some glyco-collagens, e.g. collagen VI, VII or other collagens in secondary tumours, depending on their origin, like collagens I, II, III, V and other minor collagens. The matrix linker protein Tenascin R is replaced in GBM by Tenascin C, or can be missing altogether in secondary brain tumours. In some brain tumours, hyaluronic acid of cancer origin is usually of lower molecular weight and lower quantities than in the brain matrix, and proteoglycans are usually much less aminoglycated. Also the relative volume of ECM/cells within brain tumours is markedly less than in normal brain, meaning that there is less ECM per cell to sequester any matrix-binding drug molecule compared to normal brain parenchyma ECM, ensuring that the concentration of free, unbound polycationic drug molecules is high in this environment compared to normal brain matrix

Glioblastomas are known to over-express hyaluronases (i.e. enzymes which cut hyaluronic acid into smaller molecular units). This can decrease the amount of hyaluronic acid around the tumour because small hyaluronic acid units may be washed away. Thus, in embodiments wherein the tumour is a glioblastoma multiforme (GBM), the hyaluronic acid proportion may be equal to or below 1% in the brain ECM wet weight.

The extracellular environment of GBM tumours is typically enriched in uncharged glycoproteins, in particular, fibronectin and collagens.

The invention exploits the differences in composition and volume between normal brain ECM and ECM surrounding the tumour cells by providing a cytotoxic agent for which the chemical equilibrium between ECM-bound agent and free, unbound agent favours the ECM-bound state in normal brain ECM and the free, unbound (active drug) state in the tumour ECM. A significant portion of the cytotoxic agent which is contacted with (e.g. injected into) the tumour or which is applied to the site of the resected tumour is therefore free (i.e. not bound to the abnormal ECM) to kill tumour cells in the tumour or site of the resected tumour. Any cytotoxic agent which is found beyond the surroundings of the tumour will be in contact with normal brain ECM and hence a large proportion of the cytotoxic agent will bind to the large volumes of hyaluronic acid and other negatively-charged molecules in the normal brain ECM, i.e. the combination of large volume of hyaluronic acid and strong binding between negatively-charged ECM hyaluronic acid and positively-charged cytotoxic agent ensures that the chemical equilibrium strongly favours the cytotoxic-ECM bound state. Consequently, the cytotoxic agent will essentially only exert its cytotoxic effect on the tumour cells in the tumour or at the site of the resected tumour, where the net quantity of hyaluronic acid and other negatively-charged ECM molecules (primarily proteoglycans) is low, and so the chemical equilibrium favours the unbound (active drug) state.

The brain tumour may be characterised by the composition of extracellular matrix (ECM) of the immediate environment of the brain tumour. In one embodiment, the brain tumour is one wherein the hyaluronic acid proportion of the ECM in the immediate environment of the brain tumour is equal to or below 3 wt % of the brain ECM wet weight. For example, the brain tumour may be one which expresses a hyaluronase. Alternatively, the brain tumour may be one in which the ECM/cell volume ratio is lower than in normal brain tissue by a factor of 5 or more.

As used herein, the term “immediate environment of the brain tumour” refers to the ECM in the region around the brain tumour which is less than 20 mm from a surface of the brain tumour or within its invasion progression.

Hyaluronic acid (HA) content may be tested for by measuring uronic acid content after hydrolysis in concentrated sulphuric acid or histochemical staining for hyaluronic acid on tumour sections [Cowman et al., 2015. Front Immunol. 6:261]. The most sensitive, specific, and accurate methods for determination of HA content are based on enzyme-linked sorbent assays.

Cancers are classified by the type of cell that the tumour cells resemble and is therefore presumed to be the origin of the tumour. These types include:
(i) Carcinoma: Cancers derived from epithelial cells. This group includes many of the most common cancers and include nearly all those in the breast, prostate, lung, pancreas and colon.
(ii) Sarcoma: Cancers arising from connective tissue (i.e. bone, cartilage, fat, nerve), each of which develops from cells originating in mesenchymal cells outside the bone marrow.
(iii) Germ cell tumour: Cancers derived from pluripotent cells, most often presenting in the testicle or the ovary (seminoma and dysgerminoma, respectively).
(iv) Blastoma: Cancers derived from immature “precursor” cells or embryonic tissue.

Brain and nervous system cancers include Astrocytoma, Brainstem glioma, Pilocytic astrocytoma, Ependymoma, Primitive neuro-ectodermal tumour, Cerebellar astrocytoma, Cerebral astrocytoma, Glioma, Medullo-blastoma, Neuroblastoma, Oligodendroglioma, Pineal astrocytoma, Pituitary adenoma, Visual pathway and hypothalamic glioma.

In one particularly preferred embodiment, the brain tumour is a primary brain cancer, preferably selected from the group consisting of glioblastoma multiforme (GBM), glioma, diffuse midline glioma, mixed glioma, astrocytoma, oligodendroglioma, medullo-blastoma, pineal region tumours, atypical teratoid rhabdoid tumour (AT/RT) and primitive neuroectodermal tumours (PNETS). Most preferably, the brain tumour is glioblastoma multiforme (GBM).

In a further particularly preferred embodiment, the tumour is a secondary tumour of any origin, which has metastasized into the brain.

The cytotoxic agent (e.g. polyamine) must be capable of killing tumour cells (preferably brain tumour cells) in the brain tumour or residual tumour cells at the site of the resected tumour. The action of the cytotoxic agent (alone) is capable of killing the tumour cells or inducing apoptosis cell lysis, or necrosis in the tumour cells. The cytotoxic agent is one which is capable of killing the tumour cells (preferably brain tumour cells) on its own, i.e. without an additional cancer-cell killing moiety.

In some embodiments, the cytotoxic agent (e.g. polyamine) is one which is capable of cell killing by depolarising the cell's plasma membrane. This causes water ingress and cell lysis/fragmentation.

Preferably, the cytotoxic agent (e.g. polyamine) is one which is capable of killing brain tumour cells and also tumour cells which are not from brain tumours, i.e. the killing effect of the cytotoxic agent is not specific to brain tumours. Preferably, the cytotoxic agent is capable of killing cancer cells independently of the genotype or phenotype of those cancer cells.

The ability of the cytotoxic agent to kill tumour cells may be determined by examining cells under a light microscope for full cell lysis to the point of no cellular structure is visible in the light microscope, or cell swelling and rupture, or fragmentation of cell into apoptotic structures, or loss of membrane integrity allowing penetration of binding agents (like DNA-binding dyes), or accumulation within the cell of vital stains (such as Trypan Blue or Presto Blue), or appearance outside the cell of apoptotic or necrotic markers, detectable by fluoresce/luminescence/immunochemical means.

Measurement of EC₅₀, the concentration of cytotoxic agent which induces a response (in this case cell death of a specified cell line) halfway between the baseline (no agent) and maximum effect after a specified exposure time and under specified conditions, e.g. cell culture media, is described in e.g. J. L. Seabaugh, Pharmaceut. Statist. 10 (2011) 128-134, with more critical review in M. Niepel et al., Curr. Protoc. Chem. Biol. 9 (2017) 55-74.

For example, the EC₅₀ of the cytotoxic agent may be measured using U87 cells suspended in PBS solution during 30-90 mins (e.g. 60 mins) of incubation. Cell death may be tested by Trypan Blue solution and live/dead cell count may be performed in a Neubauer Improved Haemocytometer Counting Chamber (from Hawksley).

In some embodiments, the cytotoxic agent (e.g. polyamine) binds strongly to hyaluronic acid and/or brain extracellular matrix proteoglycans. The effectiveness of binding of the cytotoxic agent to hyaluronic acid may be measured by mixing the equivalent concentration of the agent that has been determined to be toxic to cells in the assay above into 3 wt % hyaluronic acid gel (or higher wt % hyaluronic acid gel) then mixing cells into the hyaluronic acid-cytotoxic agent gel.

As used herein, the term “binds strongly to hyaluronic acid and/or brain extracellular matrix proteoglycans” means that the EC₅₀ concentration of the cytotoxic agent (e.g. obtained from a U87 cell toxicity assay in PBS, for example, determining live/dead cell counts with the Trypan Blue assay) is increased by 2-fold or more when tested in 3 wt % hyaluronic acid mixed with cell media and continues to be so for at least 3 days from the moment of application.

As used herein, the term “binds strongly to hyaluronic acid and/or brain extracellular matrix proteoglycans” may also mean that the EC₅₀ concentration which causes (50%) cell death for the cytotoxic agent in PBS causes less than 20% (preferably less than 10% or 5%) cell death to cells in 3 wt % hyaluronic acid.

In some preferred embodiments, the cytotoxic agent (e.g. polyamine) is one which does not bind strongly to brain ECM components which are strongly represented in the brain tumour ECM. In brain tumours, these are primarily components of basement membrane, e.g. fibronectin and collagens (e.g. collagen IV).

The effectiveness of binding of the cytotoxic agent to brain ECM components which are strongly represented in the brain tumour ECM may be measured by mixing the cytotoxic agent with a standard basement membrane material, e.g. Matrigel®. If the EC₅₀ value (e.g. obtained from a U87 cell toxicity assay in PBS, for example, using the Trypan Blue assay to quantify live/dead cells) is lower, or the same, or only up to 50% higher when tested in the Matrigel®, or other standard basement membrane material, then the cytotoxic agent does not bind strongly to brain ECM components which are strongly represented in the brain tumour ECM.

Matrigel® matrix (manufactured by Corning®) is a solubilized basement membrane preparation extracted from the Engelbreth-Holm-Swarm (EHS) mouse sarcoma, a tumour rich in extracellular matrix proteins, including Laminin (a major component), Collagen IV, heparin sulfate proteoglycans, entactin/nidogen, and a number of growth factors.

As used herein, the term “does not bind strongly to brain ECM components which are strongly represented in the brain tumour ECM” may also mean that the EC₅₀ concentration which causes (50%) cell death for the cytotoxic agent in PBS causes at least 20% (preferably at least 40%, 60% or 80%) cell death to brain tumour cells in Matrigel®.

Normal brain ECM comprises 10% hyaluronic acid and 15% proteoglycans (by dry weight); both of these compounds are negatively-charged. Hence polycations will bind strongly to normal brain ECM but will only have moderate binding to the uncharged (glyco) proteins in the tumour ECM environment.

In one embodiment, therefore, the cytotoxic agent is a polycation. Examples of suitable polycations include polyamines, positively-charged nanoparticles, and nanoparticles functionalized with polycations, including polyamines.

In one preferred embodiment, the cytotoxic agent is a polyamine, i.e. an organic compound having two or more amino groups. The polyamine used in the invention is a cytotoxic polyamine. A number of polyamines are known to be cytotoxic. The cytotoxicity of polyamines such as PAMAMs has been hypothesised to be due to their positive charge sequestering negatively-charged phospholipids from cell membranes, thus disrupting the cell membrane of tumour cells.

In low concentrations of polyamine, this effect has been utilised by biologists to aid transfection of cells or uptake of e.g. dye molecules by cells. In high concentrations, the cell membrane disruption is severe and causes cell death.

In some embodiments, the polyamine is an alkyl polyamine. In some embodiments, the polyamine has 2-10 (e.g. 2, 3, 4, 5, 6, 7, 8, 9 or 10) amine groups. Preferably, the polyamine is water soluble at a concentration which is toxic to brain tumour cells.

In some embodiments, the polyamine has the structure:

NH₂—[(CH₂)ₐ—NH]—[(CH₂)_{b}—NH]ₓ—[(CH₂)_{c}—NH]_{y}—H

wherein a, b and c are each independently 3, 4 or 5; and x and y are each independently 0 or 1.

In some embodiments, the polyamine has 1, 2, 3 or 4 amino groups, e.g. at least two primary amines, up to two secondary amines or up to two tertiary amines. The polyamine may comprise a polyamidoamine (PAMAM), e.g. PAMAM-g0.

Preferably, the polyamine is selected from the group consisting of spermine, spermidine, bis(hexamethylene)triamine (BHMTA), polyamidoamines (PAMAMs, e.g. PAMAM-g0), thermospermine, poly-L-lysine, poly-R-lysine, poly(allylamine), poly(allylamine) hydrochloride, poly(ethyleneimine), chitosan and chitosan derivatives, putrescine and cadaverine.

More preferably, the cytotoxic agent is 1,3-diaminopropane, putrescine, cadaverine, spermidine, spermine, thermospermine or bis(hexamethylene)triamine (BHMTA).

Most preferably, the cytotoxic agent is spermine or spermidine, or a derivative thereof; or spermidine or putrescine.

In some embodiments, the cytotoxic agent (e.g. polyamine) additionally comprises a targeting moiety which is specific for the brain or brain tumour to be targeted. The brain-targeting moiety may be one which helps the cytotoxic agent be retained within the brain. For example, the targeting moiety may be an antibody which specifically binds to an epitope on the brain tumour cells.

In some embodiments, the cytotoxic agent (e.g. polyamine) additionally comprises a moiety which limits the diffusion range of the cytotoxic agent. This may be done by increasing the molecular weight of the cytotoxic agent, e.g. by adding one or more polyethylene glycol (PEG) chains (PEGylation) or glycan moieties, e.g. hyaluronic acid or chitosan chains via oxidation of the glycan (e.g. by reaction with sodium iodide) and subsequent reaction of the oxidized glycan chain with the appropriate polyamine.

The composition comprising the cytotoxic agent (e.g. polyamine) may additionally comprise one or more additional pharmaceutically-acceptable diluents, excipients or carriers. The composition may comprise one or more of the cytotoxic agents, as defined herein. For example, the composition may comprise 1, 2, 3 or 4 different cytotoxic agents, as defined herein. The composition may also comprise one or more other active components, for example an anti-cancer agent or cancer cell-killing moiety. For example, the composition may additionally comprise one or more components selected from the group consisting of a buffer, a detergent, an inhibitor of glutathione metabolism (e.g. butionine sulfoximine), an inhibitor of amine oxidases, a proteinase inhibitor, a metalloprotease inhibitor, a hyaluronase inhibitor, an osmolite (e.g. NaCl, mannitol, etc), and a viscosity modifier.

In some embodiments, the composition does not comprise an additional cancer-cell killing moiety (i.e. the cytotoxic agent is capable of killing the brain tumour cells alone). In some embodiments, the composition does not comprise an additional anti-cancer agent (i.e. the cytotoxic agent is capable of killing the brain tumour cells alone).

The composition preferably comprises an effective amount of the cytotoxic agent (or cytotoxic reagents). As used herein, the term “effective amount” is an amount which is sufficient to kill all or substantially all (e.g. at least 70%, 80%, 90 or 95%, compared to a control without the cytotoxic agent) of the brain tumour cells in the tumour or site of the resected tumour. Effective amounts of each cytotoxic agent may readily be determined by those of skill in the art.

The structure and/or concentration of the cytotoxic agent (e.g. polyamine) in the composition is/are selected such that the chemical equilibrium between:
(i) cytotoxic agent bound to ECM and
(ii) free, unbound cytotoxic agent
   favours the ECM-bound state in normal brain ECM, and favours the free, unbound state in the tumour ECM.

The concentration of the cytotoxic agent (e.g. polyamine) in the composition is preferably 100 μM to 50 mM, e.g. 100 μM to 1 mM, 1 mM to 10 mM, or 10 mM to 50 mM. In some embodiments, the concentration of the cytotoxic agent (e.g. polyamine) is 1-50 mM or 1-25 mM, e.g. 1-5 mM, 5-10 mM, 10-15 mM, 15-20 mM, 20-25 mM, 25-30 mM, 30-35 mM, 35-40 mM, 40-45 mM or 45-50 mM. In some embodiments, the concentration of the cytotoxic agent (e.g. polyamine) is 4-25 mM. In some embodiments, the concentration of the cytotoxic agent (e.g. polyamine) is 6-15 mM, 1-12 mM, 4-12 mM or 2-10 mM.

Spermidine is toxic in high concentrations to brain tumour cells. If high concentrations of spermidine are injected into the brain, normal (healthy) areas of the brain are protected from spermidine toxicity because the spermidine binds to the hyaluronic acid and other polyanions in these areas. In the low-hyaluronic acid environment of the brain tumour, spermidine exerts its toxic effect and kills brain tumour cells. The concentration of spermidine in the composition of the invention is preferably 1 mM to 50 mM.

Particularly-preferred polyamines and their concentrations are given in the table below:

| | | |
|---|---|---|
| | Preferred | Most preferred |
| Polyamine | concentration | concentration |

| | | | | |
|---|---|---|---|---|
| Spermidine | 2-12 | mM | 4-12 | mM |
| Putrescine | 2-12 | mM | 4-10 | mM |
| Cadaverine | 1-6 | mM | 4-6 | mM |
| 1,3-diaminopropane | 2-12 | mM | 4-6 | mM |
| Spermine | 2-10 | mM | 4 | mM |
| BHMTA | 1-6 | mM | 4 | mM |

The structures and concentrations of the polyamines in the above table are, inter alia, ones which provide a chemical equilibrium between:
(i) cytotoxic agent bound to ECM and
(ii) free, unbound cytotoxic agent
   which favours the ECM-bound state in normal brain ECM, and which favours the free, unbound state in the tumour ECM (preferably wherein the tumour is a GBM tumour).

The cytotoxic agent (e.g. polyamine) is directly contacted (i.e. directly applied) in a volume which is sufficient to contact all or substantially all of the brain tumour cells in the brain tumour or at the site of the resected brain tumour. The cytotoxic agent (e.g. polyamine) may initially contact all or substantially all of the exposed brain tumour cells in the brain tumour or at the site of the resected brain tumour; the cytotoxic agent (e.g. polyamine) may then diffuse within the tumour environment to contact all brain tumour cells in the tumour.

The volume of the composition comprising the cytotoxic agent may, for example, be 50 μL to 150 mL, e.g. 50 μL-100 μL, 100 μL-500 μL, 500 μL-1 mL, 1 ml-5 mL, 5 ml-10 ml, 10 mL-50 ml, or 50 mL-150 mL. More specifically, for a 1.5 cm diameter brain tumour a volume of approximately 15 ml may be used; for a 3 cm radius brain tumour, a volume of approximately 115 mL may be used.

Step (a) refers to contacting (preferably directly contacting) the brain tumour cells in the brain tumour or the site of the resected brain tumour with a composition comprising a cytotoxic agent (e.g. polyamine).

The composition may be applied to some or all of the brain tumour cells in all or part of:
(i) the brain tumour;
(ii) the vicinity of the brain tumour;
(iii) the site of the resected brain tumour; and/or
(iv) the vicinity of the site of resected brain tumour.

The composition may be applied directly or indirectly to some or all of the above. For example, the composition may be applied directly in situ. For example, the composition may be applied by infusion using a syringe, infusion from a gel or other carrier material, by spraying or by swabbing. Alternatively, the composition may be applied indirectly. For example, the composition may be applied in the vicinity of the tumour or site of the resected tumour (e.g. 1-50 mm from any surface of the tumour or site of the resected tumour), wherein the composition diffuses into the tumour or the tumour margins or site of the resected tumour.

Preferably, the composition is applied before, during or after a surgical step to remove (resect) all or part of the tumour. The site of the resected tumour may still contain some brain tumour cells. In some embodiments, the composition is applied before a surgical step to remove all or part of the tumour. In this case, the composition may be administered systemically into the subject, wherein the agent comprises a targeting moiety which is specific for the tumour in question.

In some embodiments, the composition is applied before a surgical step to remove (resect) all or part of the tumour. In this case, the composition may be applied directly to all or part of the tumour and/or all or part of the vicinity of the tumour.

In some embodiments, the composition is applied one or more times during a surgical step to remove (resect) all or part of the tumour. In this case, the composition may be applied directly to all or part of the tumour or all or part of the vicinity of the tumour or the site of the former tumour.

In some embodiments, the composition is applied after a surgical step to remove all or part of the tumour. In this case, the composition may be applied directly to all or part of the former site of the tumour or all or part of the vicinity of the former site of tumour.

Alternatively or additionally, the composition may be administered systemically into the subject after removal of the tumour, wherein the agent comprises a targeting moiety which is specific for the tumour to be removed.

Preferably, the composition is administered topically into the tumour cavity after removal of the tumour.

In a particularly-preferred embodiment of the invention, the brain tumour is glioblastoma multiforme and the cytotoxic agent is a polyamine, for example, spermidine or spermine. Most preferably, the cytotoxic agent is applied directly to the former site of the tumour, following resection of all or part of the tumour.

Preferably, the method steps are carried out in the order specified.

The disclosure of each reference set forth herein is specifically incorporated herein by reference in its entirety.

### BRIEF DESCRIPTION OF THE FIGURES

FIG. 1. Comparative acute toxicity of selected linear polyamines towards cancer cell line of neuroepithelial origin. U87 cells were plated in 24-well plates and grown for about 3 days in liquid medium up to 50% confluency. Cell death was assayed via CellToxGreen (Promega) fluorescence after 60 minutes incubation with a polyamine and normalised to control in a lysis buffer. EC₅₀ varied from 6 mM (cadaverine) to 13-14 mM (spermidine). (B) Bright field images of U87 cell suspensions showed that cell death occurred due to severe membrane disruption resulting in cell fragmentation within 60 minutes of incubation with 10 mM and 20 mM spermine (multiplication 5×, field view: 2500 μm×1800 μm). Similar effects were found for the other polyamines studied.

FIG. 2. Proteomics showed remarkable differences in bulk extracellular matrix composition between neuroepithelial cancer cells and healthy brain. (A) U87 cells were plated in Petri dishes and grown for about 21 days in liquid medium until matrix started to peel off, then harvested. (B) Fresh calf brains were purchased from a butcher; majority of blood vessels and cerebellum was removed. Total protein was pelleted with TCA after grinding in liquid nitrogen. TCA was removed by acetone and proteins dissolved back into 9M urea. Urea-soluble proteins were separated by 1D SDS electrophoresis and identified by LC MS/MS.

FIG. 3. Neutralisation of polyamine toxicity by matrix polyanions assayed in vitro. U87 cells were suspended at 5K cells per spheroid and then grown in medium in non-adherent plates for 7-10 days until seeded into 2% HA gel pre-mixed with a polyamine for 24 hours. (A) Representative microscopic images (multiplication 5×, field view: 1800 μm×2500 μm) were taken on day 2 after spheroid was seeded into the gel. (B) Area of invasion was determined by measurement of invasion distance from the centre of four independent spheroids in four directions on days 1, 2 and 3. Standard error is provided for every point, but is sometimes not visible.

FIG. 4. In vitro HA protects the cells from polyamine toxicity when they are distant from the point of polyamine injection. Adherent cells were grown in 24-well plates in 3% HA gel until achieved 40-50% confluency. Non-toxic fluorescent dye—CellToxGreen (Promega)—was premixed into the gel. 30 μL of polyamine ( 1/20 of total gel volume) was injected at 6 o'clock position of the well. CellToxGreen penetrated only into the dead cells with compromised membranes, bound to their DNA and emitted green fluorescence, i.e. increase in fluorescence intensity quantified cell death. Cells were monitored for 7 days using well scanning functions of a fluorescent reader FLUOstar (BMG Labtech, Germany). (A) Drawing of a well to show the point of injection of polyamine and the fate of cells near the injection point; and a representative image of a well assembled from 44 scanned patches. The higher the fluorescence intensity of a patch, the higher was the number of dead cells. (B) Ratio of dead cell fluorescence in two opposite areas of the well. Ratio=[fluorescence intensity in 22 patches adjacent to the injection point]/[fluorescence intensity in 22 patches opposing the injection point]. Fluorescence intensity was averaged from four independent wells per concentration point and standard error did not exceed 15% of the intensity value. (C) After 2 days of incubation with spermine, a border between dead and live cells was formed (left picture) with round cells being dead and stained dark by Trypan Blue (central picture) and elongated cells alive and translucent (right picture). Multiplication 5×, field view: 2500 μm×1800 μm.

FIG. 5. Weight gain and survival of NCr nu/nu mice after a single polyamine injection into the brain. 6 animals per group were randomised on day 0, and on day 1 injected with 10 μL of 4 and 10 mM of the following three polyamines: putrescine, spermidine and cadaverine. Animal weight, survival and behaviour was monitored for 14 days. Only 10 mM cadaverine demonstrated acute toxicity, other polyamines and concentrations were safe and did not affect animal behaviour or weight gain.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: High Concentrations of Polyamines are Cytotoxic to Cells in Media

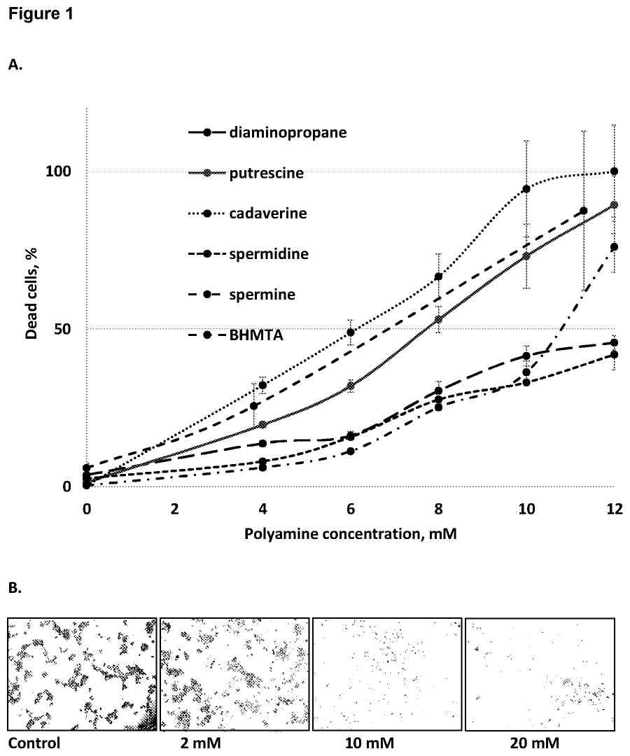

We first tested the cytotoxicity of different polyamines to brain cancer cells in suspension in media and verified that cell death occurred by membrane disruption using phase contrast optical microscopy. We tested polyamine toxicity to U87 cells, an invasive cancer cell line of neuroepithelial origin, when they were plated and incubated in full MEM medium, containing 10% foetal bovine serum. FIG. 1 shows that at the concentrations employed, all polyamines tested were highly toxic and caused severe cell plasma membrane destruction.

### Example 2: The Extracellular Matrix of Normal Brain and Brain Tumour are Significantly Different

Proteomic studies showed that invasive cancer cell line of neuroepithelial origin produced matrix which consisted mainly of proteins lacking glycosaminoglycan (GAG) modifications, i.e. lacking polyanion modifications, but contained primarily glycoproteins such as tenascin-C, collagen VI and fibronectin. In contrast, brain extracellular matrix consisted of proteoglycans, such as versican, brevican, neurocan, HAPLIN1, 2 and 4, i.e. was enriched in GAGs. Previous studies have shown that concentration of hyaluronic acid (HA), a free polyanion, in brain is around 3% w/w.

In detail, U87 cells were plated in Petri dishes and grown for about 21 days in liquid medium until the matrix produced by the cells started to peel off the Petri dish. The ECM was then harvested as a model of brain tumour ECM. Fresh calf brains, as a model of normal brain ECM, were purchased from a butcher; the majority of blood vessels and cerebellum were removed. Total protein was pelleted with TCA after grinding in liquid nitrogen for both in vitro and ex vivo samples. TCA was removed by acetone and proteins dissolved back into 9M urea. Urea-soluble proteins were separated by 1D SDS electrophoresis and identified by LC MS/MS.

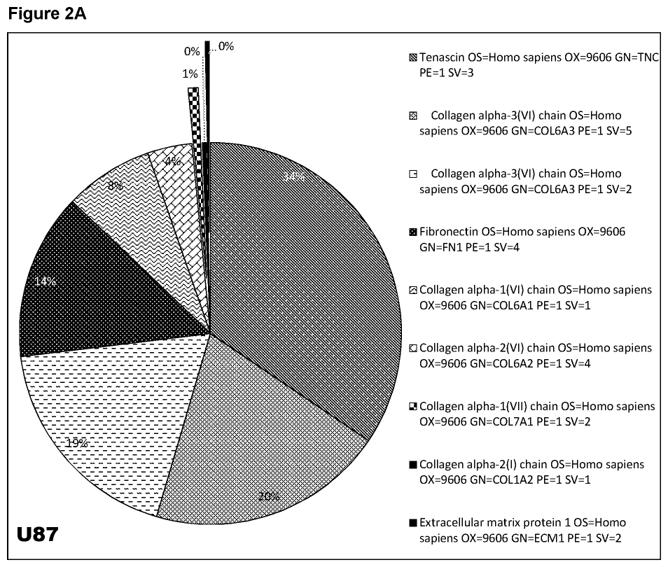

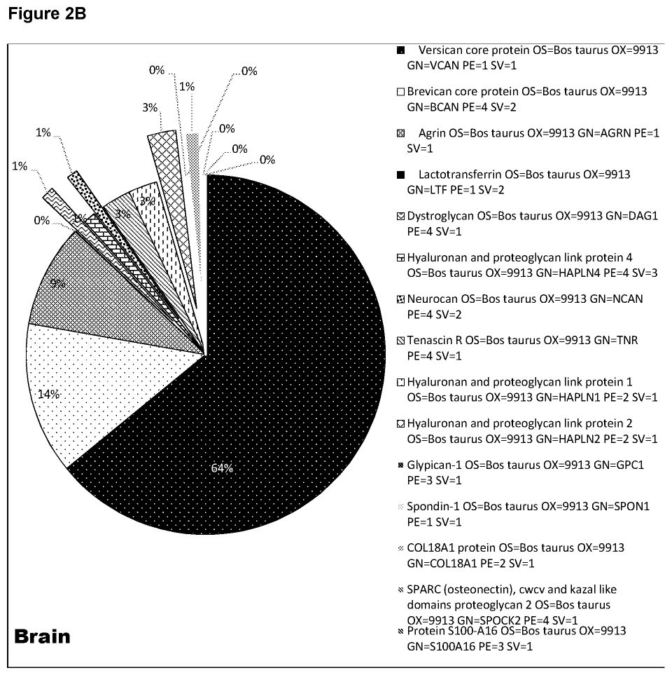

The resulting proteomics data is presented in FIG. 2 and shows a remarkable difference between the compositions of normal brain and brain tumour ECM. Thus, it is feasible to develop drugs that bind strongly to normal brain matrix and much less strongly to brain tumour ECM, so that there is a high concentration of free active drug within the tumour with few side-effects in normal brain tissue.

### Example 3: A Model of Normal Brain Matrix Abrogates the Cytotoxicity of High Concentrations of Polyamines if the Polyamine Binds Strongly to the Matrix

Strength of polyamine binding to polyanions (hyaluronic acid, GAGs) in (normal) brain ECM is a decisive factor which targets polyamine toxicity towards cancer cells rather than brain cells. When binding is strong, a polyamine will be neutralised before reaching cellular membrane which is surrounded by extracellular polyanions. When binding is weak, sufficient concentration of polyamine may kill the cell even though the cell is surrounded by polyanions. To test the binding strength of different polyamines, we developed a 3D neutralisation assay: the invasion from a neuroepithelial cancer spheroid (U87 cell line) was monitored in vitro in 2 wt % HA gel pre-mixed with different polyamines. Invasion from spheroid was indicative of cell survival and thus predictive of a strong binding of the polyamine to brain polyanions in vivo. A weak in vitro binding of a polyamine to HA manifested itself by the absence of invasion, i.e. cell poisoning, and thus was predictive of weak binding of the polyamine to brain polyanions in vivo.

Spheroids which displayed no invasion were additionally tested for metabolic activity via Presto Blue metabolic dye and found to be completely metabolically inactive, so most likely dead. Clear differences in the cell spread from the spheroids was observed with spermidine being the strongest binder to HA (invasion from spheroid was not affected by the polyamine) and spermine being the weakest binder (invasion was strongly suppressed).

In detail, polyamines were mixed with 2 wt % hyaluronic acid mixed with full MEM medium containing 10% foetal bovine serum to support cell growth to achieve the desired polyamine concentration and the resulting hyaluronic acid/polyamine gel plated in 24-well plates. One 3D tumour spheroid of U87 GBM cells was placed in each well. The invasion into the surrounding hyaluronic acid-rich matrix was imaged over several days and the invasion area measured by determining the maximum invasion distance in four orthogonal directions from the centre of the tumour spheroid and compared to controls with no polyamine in the hyaluronic acid matrix.

We found that the cytotoxicity observed for spermidine and putrescine in media is almost completely abrogated for cells in a hyaluronic acid-rich matrix. In contrast, concentrations of spermine, BHMTA and cadaverine above ˜4 mM show cytotoxic effects in the hyaluronic acid-rich matrix, suggesting that these polyamines do not bind sufficiently well to hyaluronic acid to mitigate their destructive effects on cell membranes at concentrations of polyamine>4 mM.

### Example 4: Discriminative Cytotoxicity of Polyamines to Cells in a Model of Normal Brain Matrix from an Injection of Polyamine

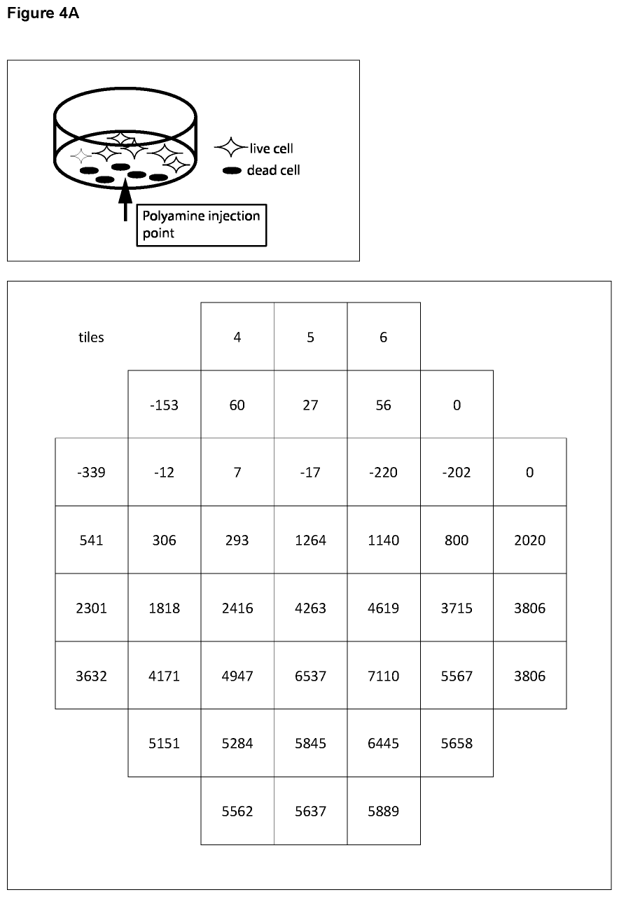

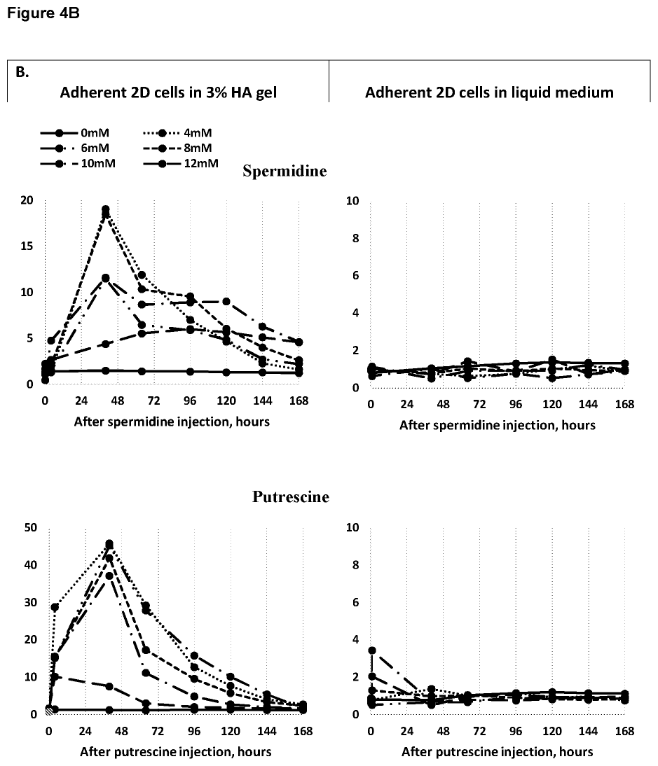

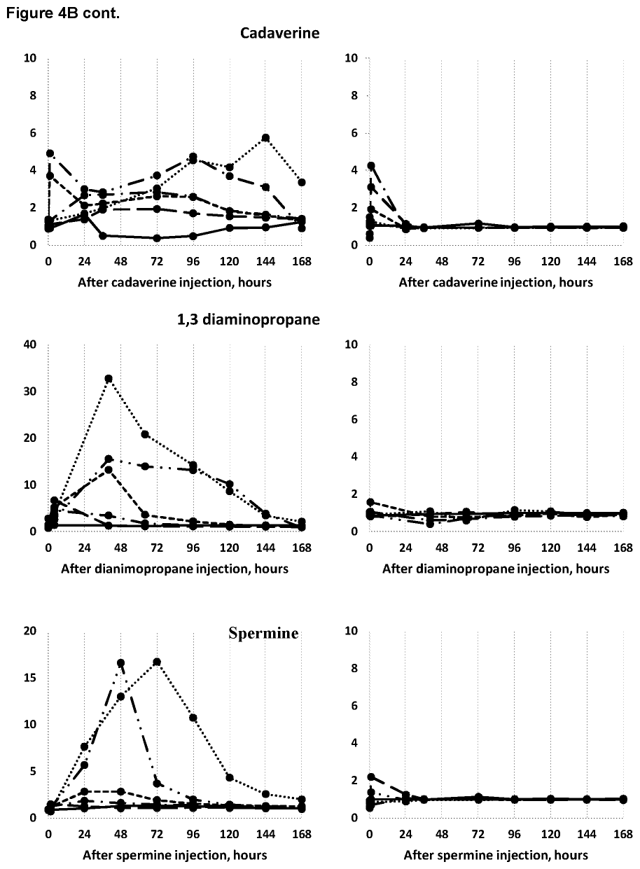

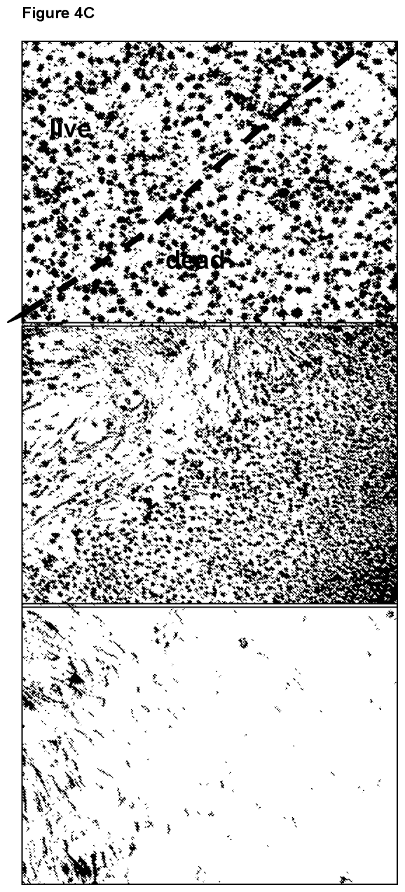

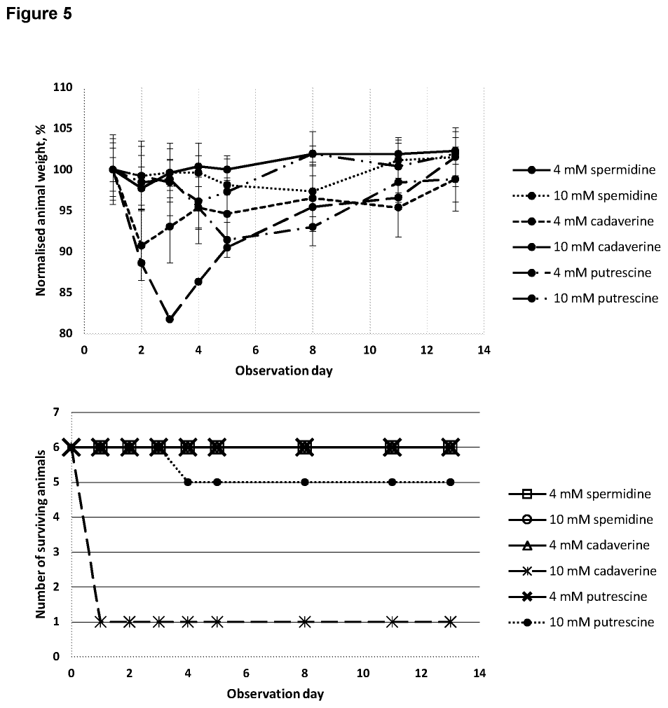

When brain cancers develop, it is within the polyanion (hyaluronic acid and GAG)-enriched environment of normal brain matrix, which in the tumour vicinity is degraded, diluted by oedema while the tumour itself synthesizes new specialized matrix with very different composition to normal brain (see Example 2). In order to demonstrate in vitro that direct injection of polyamine into a brain tumour can eliminate only the cells in vicinity of the injection, but does not affect the cells distant from the point of injection, i.e. healthy brain cells would be protected by the polyanion-rich brain ECM in vivo as shown in Example 3, we developed a 2D-scanning cell toxicity assay. First, a cancer cell line of the neuroepithelial origin (U87) was plated, given a day for the cells to adhere to the well bottom and then covered by HA gel as a model of the polyanion-rich, normal brain ECM. Then, a polyamine was injected at the 6 o'clock position of each well (see FIG. 4). In the vicinity of the injection point, cells were expected to die, because the initial polyamine concentration was maximal in this region and the HA gel in this region was diluted by the carrier solvent (PBS). However, cells distant from the injection point were still surrounded by undiluted HA and the HA gel was expected to sequester the polyamine as it diffused through the HA gel, providing the polyamine binds strongly to HA and there is sufficient HA (i.e. the HA is not diluted) to bind sufficient polyamine to reduce the effective polyamine concentration to below the EC₅₀ value. Thus, cells more distant from the injection point were expected to be protected from the polyamine, for as long as the polyamine remained strongly bound to the HA gel. Cell death was imaged over the well by scanning the well bottom for the fluorescence of a mortality dye, which binds DNA of cells with impaired membrane integrity.

The level of protection by HA of distant cells was found to be different for different polyamines (highest for putrescine and lowest for cadaverine; FIG. 4), but there was always a level of protection: for at least two days and up to seven days (the end of experiment) cell death away from the point of injection was much lower than near the injection point. In HA-depleted liquid medium cell death (controls) was evenly distributed in the well except for during the first hour after the injection.

In detail, U87 glioblastoma cells were grown in 24-well plates at with full MEM medium containing 10% foetal bovine serum until a layer of confluent cells covered the bottom of each well (FIG. 4). This liquid media was then removed leaving a layer of cells adhered to the bottom of each well. Those cells were then covered with 3 wt % hyaluronic acid hydrogel (comparable with the wt % of hyaluronic acid in human adult brain) mixed with full MEM medium containing 10% foetal bovine serum to support cell growth and CellTox™ Green Dye (Promega). This hyaluronic acid plus protein gel represents a model of human brain extracellular matrix. CEIIToxGreen is a non-toxic molecule that can only enter dead cells, where it binds to DNA and emits green fluorescence. CEIIToxGreen thus allowed us to follow cell death over time in this assay by monitoring green fluorescence.

30 μL of different concentrations of polyamine was injected at the 6 o'clock position of each well and mapped the dead cell content in each well as a function of time. Specifically, each well was mapped as 44 congruent, contiguous tiles (FIG. 4A) and cell death in a tile was assayed as the net green fluorescence intensity from the tile. As expected, tiles close to the 6 o'clock position of each well showed high levels of cell death from the first measurement time point (24 hours) and continued to rise until the end of the experiment (144 hours). In contrast, the extent of cell death in the tiles around the 12 o'clock position in each well, i.e. on the opposite side of the well from where spermine was injected, was 2 to 7-fold less than in tiles around the polyamine injection site, even after 144 hours.

Concentrations of spermidine between 4-12 mM (FIG. 4B) which we had found to be non-toxic in 2 wt % hyaluronic acid in Example 3 was found to be highly toxic for U87 cells close to the spermidine injection site in each well plate with cells dying rapidly (48-72 hours for spermidine concentrations of 4-12 mM). However, cells on the opposite side of each well plate remained viable for at least 7 days (FIG. 4). These results imply that spermidine remains highly cytotoxic to GBM cells around the location where it is injected, but that the diffusion rate and local concentration of free spermidine can be strongly limited by its binding with hyaluronic acid. The combination of these two effects is that the local concentration of spermidine around the 12 o'clock position in each well never reaches a high enough value for sufficient cell damage to cause cell death.

Examining the well plates with a phase contrast microscope upon Trypan Blue staining revealed a distinct boundary being formed between live (elongated morphology, translucent upon staining) and dead (rounded morphology, dark blue upon staining) cells (FIG. 4C). Such a boundary is consistent with our interpretation of the dead cell mapping.

We also found that concentrations of spermine of 4-6 mM whilst toxic to cells close to the injection point were benign to cells positioned just a few millimetres away from the injection point (FIG. 4B). Even 10 mM spermine was 3-fold less toxic for cells in the hyaluronic acid-rich environment compared to the same cell type in medium. The reduced toxicity of spermine here compared to Example 3 can be explained by the higher hyaluronic acid concentration used in this Example (3 wt %) compared with Example 3 (2 wt %) and confirms that polyamine cytotoxicity depends on the extent to which the polyamine can be sequestered by the extracellular matrix.

### Example 5: Cytotoxicity of Polyamines to Brain Tissue In Vivo

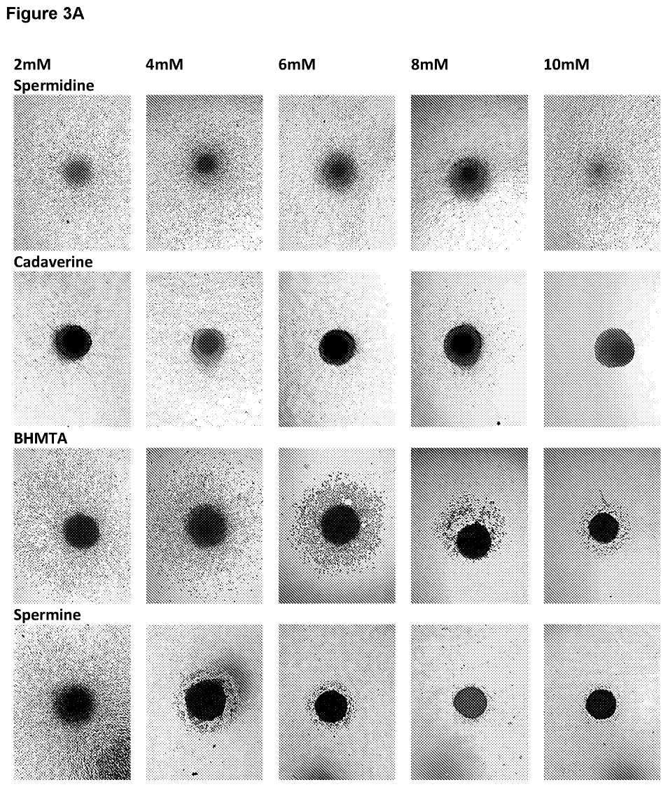

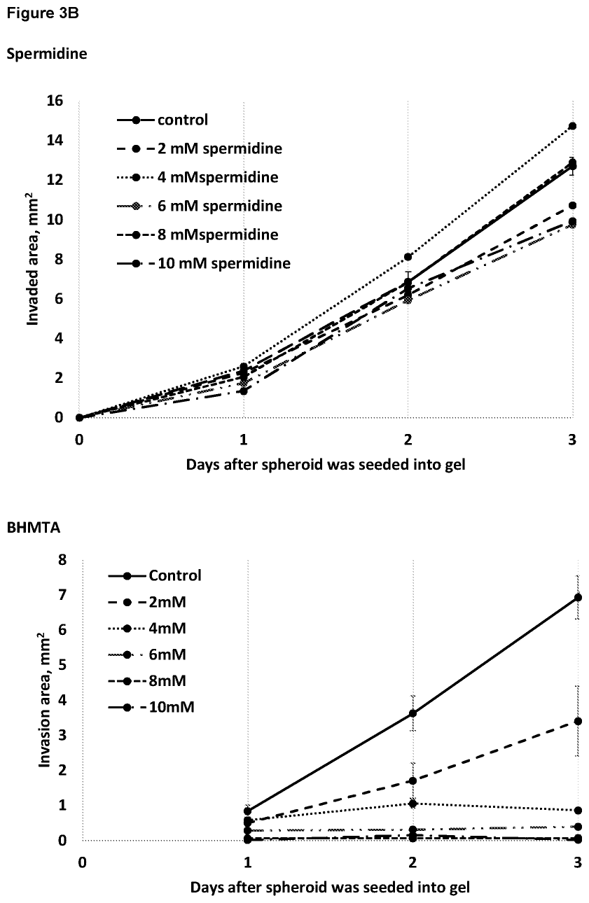

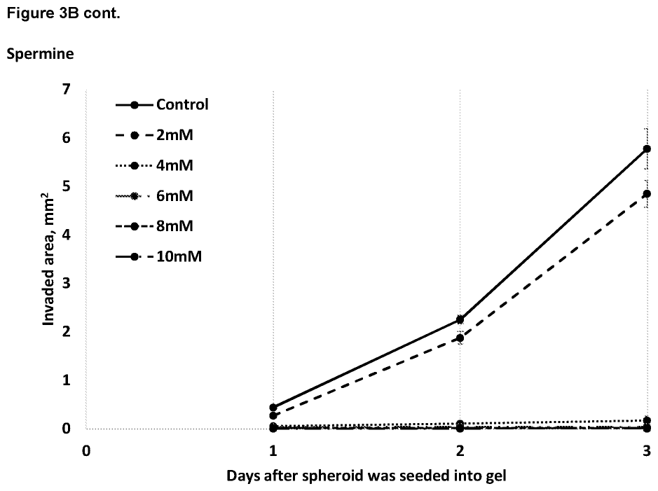

To test polyamine safety in vivo and assess their effect on brain function, a single injection of 10 μL into NCr nu/nu mouse brain was selected as a model. A 10 μL volume in a mouse brain correlates well with a 3 cm diameter tumour in human brain, which is a typical size, for example, for initial glioblastoma diagnosis. The injected volume is thus similar to what might be needed to be injected into a human brain tumour. Three polyamines were selected for testing: putrescine, spermidine and cadaverine. Polyamines were tested in two concentrations: the lowest concentration, 4 mM, was expected to be safe based on in vitro assays and the highest concentration, 10 mM, to show some toxicity for cadaverine at least. All polyamines demonstrated some toxicity at 4 mM concentration in HA-depleted medium (FIG. 1), but relatively high binding strength to HA (FIG. 3) and long-lasting protection for distant cells in HA gel (FIG. 4). All polyamines showed significant toxicity at 10 mM concentration; the EC₅₀ value for the polyamines tested is between 6 mM and 15 mM depending on the specific polyamine (FIG. 1). In our in vitro work (see Example 4, FIG. 4), cells in HA gel distant from a putrescine and spermidine injection were protected from the toxic effects of the polyamine at both 4 mM and 10 mM polyamine concentration, but not protected from 10 mM cadaverine injection. Thus, we expected cadaverine to be toxic in vivo at the high (10 mM) concentration, but not putrescine or spermidine. The in vivo experiment confirmed that 10 mM cadaverine was acutely toxic: 3 out of 6 animals died after 30 min of injection, and another 2 within 24 hours. Only one animal survived and after 18% weight loss registered on day 3, it fully recovered and gained weight very quickly. Neither putrescine nor spermidine demonstrated acute toxicity at either concentration, and 4 mM cadaverine was also safe.

Long term, no odd behavioural or clinical observations were recorded for animals treated with polyamines, so polyamine injection was found to be safe for brain function. The number of animals treated with either concentration of polyamines was sufficient to conclude that polyamines can be sufficiently neutralised by HA in brain matrix to have no effect on animal growth and behaviour long term, except 10 mM cadaverine, which was highly toxic as expected from our in vitro work (FIGS. 3 and 4). Thus, our in vitro experiments (Examples 3 and 4) show a good level of prediction and translation into a ‘real brain’ situation.

1. An in vivo method of killing brain tumour cells in a brain tumour or at a site of a resected brain tumour in a subject, the method comprising the step of:
(a) directly contacting brain tumour cells in the brain tumour or at the site of the resected brain tumour with a composition comprising a cytotoxic agent,
wherein the cytotoxic agent is a polyamine, and wherein the chemical equilibrium between:
(i) cytotoxic agent bound to ECM and
(ii) free, unbound cytotoxic agent
favours the ECM-bound state in normal brain ECM, and favours the free, unbound state in the tumour ECM.

2. (canceled)

3. (canceled)

4. The method as claimed in claim 1,
wherein the cytotoxic agent is a polyamine which binds to hyaluronic acid and/or to brain extracellular matrix proteoglycans.

5. The method as claimed in claim 1, wherein the polyamine is one wherein the EC₅₀ concentration is increased by 2-fold or more when tested in 3 wt % hyaluronic acid mixed with cell media and continues to be so for at least 4 days from the moment of application.

6. The method as claimed in claim 1, wherein the polyamine is one wherein the EC₅₀ concentration which causes cell death for the polyamine in PBS causes less than 20% cell death to cells in 3 wt % hyaluronic acid/PBS.

7. The method as claimed in claim 1, wherein the polyamine is one wherein the EC₅₀ concentration is lower, or the same, or only up to 50% higher when tested in Matrigel® (optionally in PBS).

8. The method as claimed in claim 1, wherein the polyamine is one wherein the EC₅₀ concentration which causes cell death for the polyamine in PBS causes at least 20% cell death to brain tumour cells in Matrigel® (optionally in PBS).

9. The method as claimed in claim 1, wherein the polyamine has the structure:
NH₂—[(CH₂)ₐ—NH]—[(CH₂)_{b}—NH]ₓ—[(CH₂)_{c}—NH]_{y}—H
wherein a, b and c are each independently 3, 4 or 5; and x and y are each independently 0 or 1.

10. The method as claimed in claim 1, wherein:
(a) the polyamine has 2, 3, 4, 5, 6, 7, 8, 9 or 10 amine groups; or
(b) the polyamine has at least two primary amines, up to two secondary amines or up to two tertiary amines.

11. The method as claimed in claim 1, wherein the cytotoxic agent is selected from the group consisting of spermine, spermidine, bis(hexamethylene)triamine (BHMTA), polyamidoamine (PAMAMs, e.g. PAMAM-g0), thermospermine, poly-L-lysine, poly-R-lysine poly(allylamine), poly(allylamine) hydrochloride, poly(ethyleneimine), chitosan and chitosan derivatives, putrescine and cadaverine.

12. The method as claimed in claim 1, wherein the cytotoxic agent is spermine, putrescine, cadaverine, 1,3-diaminopropane or spermidine.

13. The method as claimed in claim 1, wherein the concentration of the cytotoxic agent in the composition is 100 μM to 50 mM.

14. The method as claimed in claim 1, wherein the brain tumour is one wherein the hyaluronic acid proportion of the ECM in the immediate environment of the brain tumour is equal to or below 3 wt % of the brain ECM wet weight.

15. The method as claimed in claim 1, wherein the brain tumour is selected from the group consisting of an Astrocytoma, Brainstem glioma, Pilocytic astrocytoma, Ependymoma, Primitive neuro-ectodermal tumour, Cerebellar astrocytoma, Cerebral astrocytoma, Glioma, Medullo-blastoma, Neuroblastoma, Oligodendroglioma, Pineal astrocytoma, Pituitary adenoma, Visual pathway and a hypothalamic glioma.

16. The method as claimed in claim 1, wherein the brain tumour is selected from the group consisting of glioblastoma multiforme (GBM), glioma, diffuse midline glioma, mixed glioma, astrocytoma, oligodendroglioma, medulloblastoma, pineal region tumours, atypical teratoid rhabdoid tumour (AT/RT) and primitive neuroectodermal tumours (PNETS).

17. The method as claimed in claim 16, wherein the brain tumour is a glioblastoma multiforme (GBM).

18. The method as claimed in claim 1, wherein the cytotoxic effect of the cytotoxic agent is not specific to brain tumour cells.

19. The method as claimed in claim 1, wherein the cytotoxic agent additionally comprises:
(i) a targeting moiety which is specific for the brain or brain tumour to be targeted; or
(ii) a moiety which limits the diffusion range of the cytotoxic agent.

20. (canceled)

21. The method as claimed in claim 1, wherein the composition is applied to some or all of the brain tumour cells in all or part of:
(i) the brain tumour;
(ii) the vicinity of the brain tumour;
(iii) the site of the resected brain tumour; and/or
(iv) the vicinity of the site of resected brain tumour.

22. The method as claimed in claim 1, wherein the composition is applied by infusion using a syringe, infusion from a gel or other carrier material, by spraying or by swabbing.

23. The method as claimed in claim 1, wherein the brain tumour is glioblastoma multiforme and the cytotoxic agent is spermidine, spermine or putrescine.
